(19) United States
(12) Patent Application Publication
(10) Pub. No.: US 2008/0234347 A1
(43) Pub. Date: Sep. 25, 2008

(54) METHOD FOR TREATING CB2 RECEPTOR MEDIATED PAIN

(76) Inventors: Mingde Xia, Belle Mead, NJ (US); Mark J. Macielag, Branchburg, NJ (US); Christopher M. Flores, Lansdale, PA (US); Kevin Paul Pavlick, New Britain, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/038,029

(22) Filed: Feb. 27, 2008

Related U.S. Application Data
(60) Provisional application No. 60/896,014, filed on Mar. 21, 2007.

(51) Int. Cl. A61K 31/416; A61P 29/00

(52) U.S. Cl. 514/405; 514/407

(57) ABSTRACT

The present invention is directed to a method for treating, ameliorating or preventing CB2 receptor mediated pain in a subject in need thereof comprising administering to the subject an effective amount of a compound of formula (I):

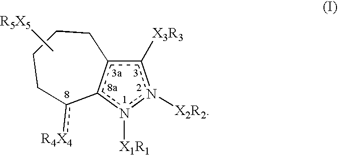

or a form thereof, wherein X₁R₁, X₂R₂, X₃R₃, X₄R⁴ and X₅R₅ are as defined herein.

### FIELD OF THE INVENTION

This invention is directed to a method for treating, ameliorating or preventing CB2 receptor mediated pain in a subject in need thereof. More particularly, said method comprises administering to the subject an effective amount of a hexahydro-cycloheptapyrazole CB2 agonist compound of the present invention.

### BACKGROUND OF THE INVENTION

PCT Application WO2006/030124 describes pyrazole derivatives as CB1 or CB2 receptor agonists.

CB2-selective agonists have been shown to be effective in the carrageenan paw model of inflammatory pain and therefore may be effective in the treatment of acute and chronic inflammatory pain (Gutierrez T, Farthing J N, Zvonok A M, Makriyannis A and Hohmann A G, Activation of peripheral cannabinoid CB1 and CB2 receptors suppresses the maintenance of inflammatory nociception: A comparative analysis, British Journal of Pharmacology, (2007), 150(2), 153-163; Quartilho A, Mata H P, Ibrahim M M, Vanderah T W, Porreca F, Makriyannis A and Malan T P, Jr., Inhibition of Inflammatory Hyperalgesia by Activation of Peripheral CB2 Cannabinoid Receptors, Anesthesiology, (2003), 99(4), 955-960; and, Nackley A G, Makriyannis A and Hohmann A G, Selective activation of cannabinoid CB2 receptors suppresses spinal Fos protein expression and pain behavior in a rat model of inflammation, Neuroscience (Oxford, United Kingdom) (2003), 119(3), 747-757).

CB2-selective agonists have also been shown to be effective inhibitors of thermal nociception in transgenic mice and potentially useful for the treatment of acute pain (Ibrahim M M, Rude M L, Stagg N J, Mata H P, Lai J, Vanderah T W, Porreca F, Buckley N E, Makriyannis A and Malan T P, Jr., CB2 cannabinoid receptor mediation of antinociception, Pain, (2006), 122(1-2), 36-42).

Activation of the CB2 receptor produces antinociception following surgical incision, suggesting that selective cannabinoid CB2 receptor agonists might be useful in the management of postoperative pain (LaBuda C J, Koblish M and Little P J, Cannabinoid CB2 receptor agonist activity in the hindpaw incision, European Journal of Pharmacology, (2005), 527(1-3), 172-174).

Activation of peripheral cannabinoid CB2 receptors are sufficient to normalize nociceptive thresholds and produce antinociception in persistent pain states (Hohmann A G, Farthing J N, Zvonok A M and Makriyannis A, Selective activation of cannabinoid CB2 receptors suppresses hyperalgesia evoked by intradermal capsaicin, Journal of Pharmacology and Experimental Therapeutics, (2004), 308(2), 446-453).

Selective CB2 receptor agonists inhibit acute, chronic, inflammatory and neuropathic pain responses in animal models and, therefore, show promise for the treatment of acute and chronic pain (Malan T P, Jr., Ibrahim M M, Lai J, Vanderah T W, Makriyannis A and Porreca F, CB2 cannabinoid receptor agonists: pain relief without psychoactive effects?, Current Opinion in Pharmacology, (2003), 3(1), 62-67; Ibrahim M M, Deng H, Zvonok A, Cockayne D A, Kwan J, Mata H P, Vanderah T W, Lai J, Porreca F, Makriyannis A and Malan T P, Jr., Activation of CB2 cannabinoid receptors by AM1241 inhibits experimental neuropathic pain: Pain inhibition by receptors not present in the CNS, Proceedings of the National Academy of Sciences of the United States of America, (2003), 100(18), 10529-10533; and, Burns T L and Ineck J R, Cannabinoid analgesia as a potential new therapeutic option in the treatment of chronic pain, Annals of Pharmacotherapy, (2006), 40(2), 251-260).

The CB2 receptor-selective agonist AM1241 produces antinociception to thermal stimuli (Malan T P, Jr., Ibrahim M M, Deng H, Liu Q, Mata H P, Vanderah T, Porreca F and Makriyannis A, CB2 cannabinoid receptor-mediated peripheral antinociception, Pain, (2001), 93(3), 239-245).

### DETAILED DESCRIPTION OF THE INVENTION

The present invention is directed to a method for treating, ameliorating or preventing CB2 receptor mediated pain in a subject in need thereof comprising administering to the subject an effective amount of a compound of formula (I):

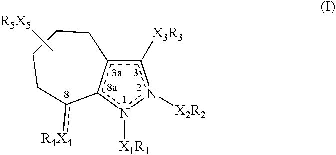

or a form thereof, wherein
the dashed lines between positions 2-3 and positions 3a-8a in formula (I) represent locations for each of two double bonds present when X₁R₁ is present;
the dashed lines between positions 3-3a and positions 8a-1 in formula (I) represent locations for each of two double bonds present when X₂R₂ is present;
the dashed line between position 8 and X₄R₄ in formula (I) represents the location for a double bond;
X₁ is absent or lower alkylene;
X₂ is absent or lower alkylene;
wherein only one of X₁R₁ and X₂R₂ are present;
X₃ is absent, lower alkylene, lower alkylidene or —NH—;
when the dashed line between position 8 and X₄R₄ is absent, X₄ is absent, or is lower alkylene;
when the dashed line between position 8 and X₄R₄ is present, X₄ is absent;
X₅ is absent or lower alkylene;
R₁ is selected from hydrogen, alkyl (optionally substituted at one or more positions by halogen, hydroxy or lower alkoxy), lower alkyl-sulfonyl, aryl, C₃-C₁₂ cycloalkyl or heterocyclyl, wherein aryl, C₃-C₁₂ cycloalkyl or heterocyclyl are each optionally substituted at one or more positions by halogen, aminosulfonyl, lower alkyl-aminosulfonyl, alkyl (optionally substituted at one or more positions by halogen, hydroxy or lower alkoxy), hydroxy or lower alkoxy (optionally substituted at one or more positions by halogen or hydroxy);
R₂ is selected from hydrogen, alkyl (optionally substituted at one or more positions by halogen, hydroxy or lower alkoxy), lower alkyl-sulfonyl, aryl, C₃-C₁₂ cycloalkyl or heterocyclyl, wherein aryl, C₃-C₁₂ cycloalkyl or heterocyclyl are each optionally substituted at one or more positions by halogen, aminosulfonyl, lower alkyl-aminosulfonyl, alkyl (optionally substituted at one or more positions by halogen, hydroxy or lower alkoxy), hydroxy or lower alkoxy (optionally substituted at one or more positions by halogen or hydroxy);
R₃ is —C(O)-Z₁(R₆), —SO₂—NR₇-Z₂(R₅) or —C(O)—NR₉-Z₃(R₁₀);
when the dashed line between position 8 and X₄R₄ is absent, X₄ is absent or lower alkylene and R₄ is hydroxy, lower alkoxy, halogen, aryl, C₃-C₁₂ cycloalkyl or heterocyclyl, wherein aryl, C₃-C₁₂ cycloalkyl or heterocyclyl are each optionally substituted at one or more positions by hydroxy, oxo, lower alkyl (optionally substituted at one or more positions by halogen, hydroxy or lower alkoxy), lower alkoxy (optionally substituted at one or more positions by halogen or hydroxy) or halogen;
when the dashed line between position 8 and X₄R₄ is present, X₄ is absent and R₄ is CH-aryl or CH-heterocyclyl, wherein aryl or heterocyclyl are each optionally substituted at one or more positions by hydroxy, oxo, lower alkyl (optionally substituted at one or more positions by halogen, hydroxy or lower alkoxy), lower alkoxy (optionally substituted at one or more positions by halogen or hydroxy) or halogen;
R₅ is hydrogen, hydroxy, oxo, halogen, amino, lower alkyl-amino, alkyl (optionally substituted at one or more positions by halogen, hydroxy or lower alkoxy), lower alkoxy (optionally substituted at one or more positions by halogen or hydroxy), carboxy, carbonylalkoxy, carbamoyl, carbamoylalkyl, aryl, aryloxy, arylalkoxy or heterocyclyl;
R₆ is aryl, C₃-C₁₂ cycloalkyl or heterocyclyl each optionally substituted by one or more hydroxy, oxo, halogen, amino, lower alkyl-amino, alkyl (optionally substituted at one or more positions by halogen, hydroxy or lower alkoxy), lower alkoxy (optionally substituted at one or more positions by halogen or hydroxy), carboxy, carbonylalkoxy, carbamoyl, carbamoylalkyl, aryl, aryloxy, arylalkoxy or heterocyclyl;
R₇ is hydrogen or lower alkyl;
R₈ is hydrogen, aryl, C₃-C₁₂ cycloalkyl or heterocyclyl, wherein aryl, C₃-C₁₂ cycloalkyl or heterocyclyl are each optionally substituted by one or more hydroxy, oxo, halogen, amino, lower alkyl-amino, alkyl (optionally substituted at one or more positions by halogen, hydroxy or lower alkoxy), lower alkoxy (optionally substituted at one or more positions by halogen or hydroxy), carboxy, carbonylalkoxy, carbamoyl, carbamoylalkyl, aryl, aryloxy, arylalkoxy or heterocyclyl;
R₉ is hydrogen or lower alkyl;
R₁₀ is hydrogen, aryl, C₃-C₁₂ cycloalkyl or heterocyclyl, wherein aryl, C₃-C₁₂ cycloalkyl or heterocyclyl are each optionally substituted by one or more hydroxy, oxo, halogen, amino, lower alkyl-amino, alkyl (optionally substituted at one or more positions by halogen, hydroxy or lower alkoxy), lower alkoxy (optionally substituted at one or more positions by halogen or hydroxy), carboxy, carbonylalkoxy, carbamoyl, carbamoylalkyl, aminosulfonyl, lower alkyl-aminosulfonyl, aryl, aryloxy, arylalkoxy or heterocyclyl;
Z₁ and Z₂ are each absent or alkyl; and,
Z₃ is absent, —NH—, —SO₂— or alkyl (wherein alkyl is optionally substituted at one or more positions by halogen, hydroxy, lower alkyl, lower alkoxy, carboxy or carbonylalkoxy).

An example of the present invention includes a compound of formula (I) or a salt, isomer, prodrug, metabolite or polymorph thereof wherein X₁ is absent and R₁ is selected from hydrogen, alkyl, lower alkyl-sulfonyl, aryl, C₃-C₁₂ cycloalkyl or heterocyclyl, wherein aryl or heterocyclyl are each optionally substituted at one or more positions by halogen, aminosulfonyl or alkyl (optionally substituted at one or more positions by halogen).

An example of the present invention includes a compound of formula (I) or a salt, isomer, prodrug, metabolite or polymorph thereof wherein R₃ is —SO₂—NR₇-Z₂(R₅); X₃ is absent or lower alkylidene; R₇ is hydrogen or lower alkyl; Z₂ is absent or alkyl; and, R₈ is aryl, C₃-C₁₂ cycloalkyl or heterocyclyl.

An example of the present invention includes a compound of formula (I) or a salt, isomer, prodrug, metabolite or polymorph thereof wherein R₃ is —SO₂—NH-Z₂(R₅); X₃ is absent or lower alkylidene; Z₂ is absent or alkyl; and, R₈ is aryl, C₃-C₁₂ cycloalkyl or heterocyclyl.

An example of the present invention includes a compound of formula (I) or a salt, isomer, prodrug, metabolite or polymorph thereof wherein R₃ is —C(O)—NR₉-Z₃(R₁₀); X₃ is absent or lower alkylidene; R₉ is hydrogen or lower alkyl; Z₃ is absent, —SO₂— or alkyl (wherein alkyl is optionally substituted at one or more positions by halogen, hydroxy or carbonylalkoxy); and, R₁₀ is hydrogen, aryl, C₃-C₁₂ cycloalkyl or heterocyclyl, wherein aryl, C₃-C₁₂ cycloalkyl or heterocyclyl are each optionally substituted by one or more hydroxy, halogen, alkyl (optionally substituted at one or more positions by halogen), alkoxy, carboxy, carbonylalkoxy, carbamoylalkyl or aminosulfonyl.

An example of the present invention includes a compound of formula (I) or a salt, isomer, prodrug, metabolite or polymorph thereof wherein R₃ is —C(O)—NH-Z₃(R₁₀); X₃ is absent or lower alkylidene; Z₃ is absent, —SO₂— or alkyl (wherein alkyl is optionally substituted at one or more positions by halogen, hydroxy or carbonylalkoxy); and, R₁₀ is hydrogen, aryl, C₃-C₁₂ cycloalkyl or heterocyclyl, wherein aryl, C₃-C₁₂ cycloalkyl or heterocyclyl are each optionally substituted by one or more hydroxy, halogen, alkyl (optionally substituted at one or more positions by halogen), alkoxy, carboxy, carbonylalkoxy, carbamoylalkyl or aminosulfonyl.

An example of the present invention includes a compound of formula (I) or a salt, isomer, prodrug, metabolite or polymorph thereof wherein R₃ is —C(O)—NH-Z₃(R₁₀); X₃ is absent or lower alkylidene; Z₃ is absent, —SO₂— or alkyl (wherein alkyl is optionally substituted at one or more positions by halogen, hydroxy or carbonylalkoxy); and, R₁₀ is aryl optionally substituted by one or more hydroxy, halogen, alkyl (optionally substituted at one or more positions by halogen), alkoxy or aminosulfonyl.

An example of the present invention includes a compound of formula (I) or a salt, isomer, prodrug, metabolite or polymorph thereof wherein R₃ is —C(O)—NH-Z₃(R₁₀); X₃ is absent or lower alkylidene; Z₃ is absent, —SO₂— or alkyl (wherein alkyl is optionally substituted at one or more positions by halogen, hydroxy or carbonylalkoxy); and, R₁₀ is hydrogen or C₃-C₁₂ cycloalkyl, wherein C₃-C₁₂ cycloalkyl is optionally substituted by one or more hydroxy, alkyl, alkoxy, carboxy, carbonylalkoxy or carbamoylalkyl.

An example of the present invention includes a compound of formula (I) or a salt, isomer, prodrug, metabolite or polymorph thereof wherein R₃ is —C(O)—NH-Z₃(R₁₀); X₃ is absent or lower alkylidene; Z₃ is absent, —SO₂— or alkyl (wherein alkyl is optionally substituted at one or more positions by halogen, hydroxy or carbonylalkoxy); and, R₁₀ is hydrogen or heterocyclyl, wherein heterocyclyl is optionally substituted by one or more carbonylalkoxy.

An example of the present invention includes a compound of formula (I) or a salt, isomer, prodrug, metabolite or polymorph thereof wherein the dashed line between position 8 and X₄R₄ is absent, X₄ is absent or lower alkylene and R₄ is aryl optionally substituted at one or more positions by lower alkyl or halogen.

An example of the present invention includes a compound of formula (I) or a salt, isomer, prodrug, metabolite or polymorph thereof wherein the dashed line between position 8 and X₄R₄ is present, X₄ is absent and R₄ is CH-aryl or CH-heterocyclyl, wherein aryl or heterocyclyl are each optionally substituted at one or more positions by lower alkoxy or halogen.

An example of the present invention includes a compound of formula (I) or a salt, isomer, prodrug, metabolite or polymorph thereof wherein X₅ is absent and R₅ is hydrogen.

An example of the present invention includes a compound of formula (Ia)

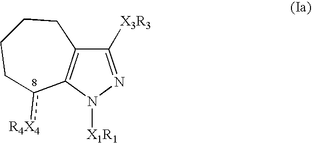

or a salt, isomer, prodrug, metabolite or polymorph thereof wherein X₁ is absent or lower alkylene; X₃ is absent or lower alkylidene; X₄ is absent or is lower alkylene when the dashed line between position 8 and X₄R₄ is absent; X₄ is absent when the dashed line between position 8 and X₄R₄ is present; R₁ is selected from hydrogen, alkyl, lower alkyl-sulfonyl, aryl, C₃-C₁₂ cycloalkyl or heterocyclyl, wherein aryl or heterocyclyl are each optionally substituted at one or more positions by halogen, aminosulfonyl or alkyl (optionally substituted at one or more positions by halogen); R₃ is —C(O)—(R₆), —SO₂—NH-Z₂(R₈) or —C(O)—NH-Z₃(R₁₀); when the dashed line between position 8 and X₄R₄ is absent, R₄ is aryl, wherein aryl is optionally substituted at one or more positions by lower alkyl or halogen; when the dashed line between position 8 and X₄R₄ is present, R₄ is CH-aryl or CH-heterocyclyl, wherein aryl or heterocyclyl are each optionally substituted at one or more positions by lower alkoxy or halogen; R₆ is heterocyclyl optionally substituted by one or more aryl or heterocyclyl; Z₂ is absent or alkyl; R₈ is aryl, C₃-C₁₂ cycloalkyl or heterocyclyl; Z₃ is absent, —SO₂— or alkyl (wherein alkyl is optionally substituted at one or more positions by halogen, hydroxy or carbonylalkoxy); and, R₁₀ is hydrogen, aryl, C₃-C₁₂ cycloalkyl or heterocyclyl, wherein aryl, C₃-C₁₂ cycloalkyl or heterocyclyl are each optionally substituted by one or more hydroxy, halogen, alkyl (optionally substituted at one or more positions by halogen), alkoxy, carboxy, carbonylalkoxy, carbamoylalkyl or aminosulfonyl.

An example of the present invention includes a compound of formula (Ia) or a salt, isomer, prodrug, metabolite or polymorph thereof wherein X₁ is absent; X₃ is absent or lower alkylidene; X₄ is lower alkylene when the dashed line between position 8 and X₄R₄ is absent; X₄ is absent when the dashed line between position 8 and X₄R₄ is present; R₁ is selected from hydrogen or alkyl; R₃ is —SO₂—NH-Z₂(R₈) or —C(O)—NH-Z₃(R₁₀); when the dashed line between position 8 and X₄R₄ is absent, R₄ is aryl, wherein aryl is optionally substituted at one or more positions by lower alkyl or halogen; when the dashed line between position 8 and X₄R₄ is present, R₄ is CH-aryl or CH-heterocyclyl, wherein aryl or heterocyclyl are each optionally substituted at one or more positions by lower alkoxy or halogen; Z₂ is absent or alkyl; R₈ is aryl or heterocyclyl; Z₃ is alkyl (wherein alkyl is optionally substituted at one or more positions by halogen, hydroxy or carbonylalkoxy); and, R₁₀ is aryl or heterocyclyl, wherein aryl or heterocyclyl are each optionally substituted by one or more hydroxy, halogen, alkyl (optionally substituted at one or more positions by halogen), alkoxy, carboxy, carbonylalkoxy, carbamoylalkyl or aminosulfonyl.

An example of the present invention includes a compound of Formula (I) and pharmaceutically acceptable forms thereof selected from:

| Cpd | Name |
|---|---|
| 1 | 8-(3-chloro-benzyl)-1,4,5,6,7,8-hexahydro-cycloheptapyrazole-3-carboxylic acid |
| | [(1S)-2-hydroxy-1-phenyl-ethyl]-amide, |
| 2 | (8R*)-8-(3-chloro-benzyl)-1,4,5,6,7,8-hexahydro-cycloheptapyrazole-3-carboxylic |
| | acid [(1R)-2-hydroxy-1-phenyl-ethyl]-amide, |
| 3 | (8R*)-8-(3-chloro-benzyl)-1,4,5,6,7,8-hexahydro-cycloheptapyrazole-3-carboxylic |
| | acid [(1S)-2-hydroxy-1-phenyl-ethyl]-amide, |
| 4 | (8S*)-8-(3-chloro-benzyl)-1,4,5,6,7,8-hexahydro-cycloheptapyrazole-3-carboxylic |
| | acid [(1S)-2-hydroxy-1-phenyl-ethyl]-amide, |
| 5 | (2E)-2-[(8R*)-8-(3-fluoro-benzyl)-1,4,5,6,7,8-hexahydro-cycloheptapyrazol- |
| | 3-yl]-ethenesulfonic acid [(1S-1-phenyl-ethyl]-amide, |
| 6 | (8E)-8-(4-chloro-benzylidene)-1,4,5,6,7,8-hexahydro-cycloheptapyrazole-3- |
| | carboxylic acid [(1R)-2-hydroxy-1-phenyl-ethyl]-amide, |
| 7 | (2E,8E)-2-[8-(4-chloro-benzylidene)-1,4,5,6,7,8-hexahydro- |
| | cycloheptapyrazol-3-yl]-ethenesulfonic acid [(1S)-1-phenyl-ethyl]-amide, |
| 8 | (8E)-(2S)-2-{[8-(4-chloro-benzylidene)-1,4,5,6,7,8-hexahydro- |
| | cycloheptapyrazole-3-carbonyl]-amino}-3-(4-fluoro-phenyl)-propionic acid |
| | methyl ester, |
| 9 | (8E)-8-(3-chloro-benzylidene)-1,4,5,6,7,8-hexahydro-cycloheptapyrazole-3- |
| | carboxylic acid [(1R)-2-hydroxy-1-phenyl-ethyl]-amide, |
| 10 | (8E)-(2S)-2-{[8-(3-chloro-benzylidene)-1,4,5,6,7,8-hexahydro- |
| | cycloheptapyrazole-3-carbonyl]-amino}-3-(4-fluoro-phenyl)-propionic acid |
| | methyl ester, |
| 11 | (8E)-(2S)-2-{[8-(3-fluoro-benzylidene)-1-methyl-1,4,5,6,7,8-hexahydro |
| | cycloheptapyrazole-3-carbonyl]-amino}-3-(4-fluoro-phenyl)-propionic acid |
| | methyl ester, |
| 12 | (8E)-8-(3-fluoro-benzylidene)-1-methyl-1,4,5,6,7,8-hexahydro- |
| | cycloheptapyrazole-3-carboxylic acid [(1R)-2-hydroxy-1-phenyl-ethyl]-amide, |
| 13 | (8E)-(2S)-8-(3-fluoro-benzylidene)-1-methyl-1,4,5,6,7,8-hexahydro- |
| | cycloheptapyrazole-3-carboxylic acid [1-hydroxymethyl-2-(4-hydroxy- |
| | phenyl)-ethyl]-amide, |
| 14 | (8E)-(2R)-2-{[8-(3-chloro-benzylidene)-1,4,5,6,7,8-hexahydro- |
| | cycloheptapyrazole-3-carbonyl]-amino}-3-(4-fluoro-phenyl)-propionic acid |
| | methyl ester, |
| 15 | (8E)-(2R)-2-{[8-(4-chloro-benzylidene)-1,4,5,6,7,8-hexahydro- |
| | cycloheptapyrazole-3-carbonyl]-amino}-3-(4-fluoro-phenyl)-propionic acid |
| | methyl ester, |
| 16 | (8E)-8-(3-fluoro-benzylidene)-1-methyl-1,4,5,6,7,8-hexahydro- |
| | cycloheptapyrazole-3-carboxylic acid [(1R)-3-hydroxy-1-phenyl-propyl]-amide, |
| 17 | (8E)-8-(3-chloro-benzylidene)-1,4,5,6,7,8-hexahydro-cycloheptapyrazole-3- |
| | carboxylic acid [(1R)-3-hydroxy-1-phenyl-propyl]-amide, |
| 18 | (8R*)-(3-chloro-benzyl)-1,4,5,6,7,8-hexahydro-cycloheptapyrazole-3- |
| | carboxylic acid [(1S)-2-methoxy-1-phenyl-ethyl]-amide, |
| 19 | (8S*)-(3-chloro-benzyl)-1,4,5,6,7,8-hexahydro-cycloheptapyrazole-3- |
| | carboxylic acid [(1S)-2-methoxy-1-phenyl-ethyl]-amide, |
| 20 | (8S*)-(3-chloro-benzyl)-1,4,5,6,7,8-hexahydro-cycloheptapyrazole-3- |
| | carboxylic acid [(1R)-2-methoxy-1-phenyl-ethyl]-amide, and |
| 21 | (8R*)-(3-chloro-benzyl)-1,4,5,6,7,8-hexahydro-cycloheptapyrazole-3- |
| | carboxylic acid [(1R)-2-methoxy-1-phenyl-ethyl]-amide. |

### DEFINITIONS

As used herein, the following terms have the following meanings:

The term “alkyl” means a saturated branched or straight chain monovalent hydrocarbon radical of up to 10 carbon atoms. Alkyl typically includes, but is not limited to, methyl, ethyl, propyl, isopropyl, n-butyl, t-butyl, pentyl, hexyl, heptyl and the like.

The term “lower alkyl” means an alkyl radical of up to 4 carbon atoms. The point of attachment may be on any alkyl or lower alkyl carbon atom and, when further substituted, substituent variables may be placed on any carbon atom.

The term “alkylene” means a saturated branched or straight chain monovalent hydrocarbon linking group of up to 10 carbon atoms, whereby the linking group is derived by the removal of one hydrogen atom each from two carbon atoms. Alkylene typically includes, but is not limited to, methylene, ethylene, propylene, isopropylene, n-butylene, t-butylene, pentylene, hexylene, heptylene and the like. The term “lower alkylene” means an alkylene linking group of up to 4 carbon atoms. The point of attachment may be on any alkylene or lower alkylene carbon atom and, when further substituted, substituent variables may be placed on any carbon atom.

The term “alkylidene” means an alkylene linking group of from 1 to 10 carbon atoms having at least one double bond formed between two adjacent carbon atoms, wherein the double bond is derived by the removal of one hydrogen atom each from the two carbon atoms. Atoms may be oriented about the double bond in either the cis (E) or trans (Z) conformation. Alkylidene typically includes, but is not limited to, methylidene, vinylidene, propylidene, iso-propylidene, methallylene, allylidene (2-propenylidene), crotylene (2-butenylene), prenylene (3-methyl-2-butenylene) and the like. The term “lower alkylidene” means a radical or linking group of from 1 to 4 carbon atoms. The point of attachment may be on any alkylidene or lower alkylidene carbon atom and, when further substituted, substituent variables may be placed on any carbon atom.

The term “alkoxy” means an alkyl, alkylene or alkylidene radical of up to 10 carbon atoms attached via an oxygen atom, whereby the point of attachment is formed by the removal of the hydrogen atom from a hydroxide substituent on a parent radical.

The term “lower alkoxy” means an alkyl, alkylene or alkylidene radical of up to 4 carbon atoms. Lower alkoxy typically includes, but is not limited to, methoxy, ethoxy, propoxy, butoxy and the like. When further substituted, substituent variables may be placed on any alkoxy carbon atom.

The term “cycloalkyl” means a saturated or partially unsaturated monocyclic, polycyclic or bridged hydrocarbon ring system radical or linking group. A ring of 3 to 20 carbon atoms may be designated by C₃₋₂₀ cycloalkyl; a ring of 3 to 12 carbon atoms may be designated by C₃₋₁₂ cycloalkyl, a ring of 3 to 8 carbon atoms may be designated by C₃₋₈ cycloalkyl and the like.

Cycloalkyl typically includes, but is not limited to, cyclopropyl, cyclobutyl, cyclopentyl, cyclohexyl, cyclohexenyl, cycloheptyl, cyclooctyl, indanyl, indenyl, 1,2,3,4-tetrahydro-naphthalenyl, 5,6,7,8-tetrahydro-naphthalenyl, 6,7,8,9-tetrahydro-5H-benzocycloheptenyl, 5,6,7,8,9,10-hexahydro-benzocyclooctenyl, fluorenyl, bicyclo[2.2.1]heptyl, bicyclo[2.2.1]heptenyl, bicyclo[2.2.2]octyl, bicyclo[3.1.1]heptyl, bicyclo[3.2.1]octyl, bicyclo[2.2.2]octenyl, bicyclo[3.2.1]octenyl, adamantanyl, octahydro-4,7-methano-1H-indenyl, octahydro-2,5-methano-pentalenyl (also referred to as hexahydro-2,5-methano-pentalenyl) and the like. When further substituted, substituent variables may be placed on any ring carbon atom.

The term “heterocyclyl” means a saturated, partially unsaturated or unsaturated monocyclic, polycyclic or bridged hydrocarbon ring system radical or linking group, wherein at least one ring carbon atom has been replaced with one or more heteroatoms independently selected from N, O or S. A heterocyclyl ring system further includes a ring system having up to 4 nitrogen atom ring members or a ring system having from 0 to 3 nitrogen atom ring members and 1 oxygen or sulfur atom ring member. When allowed by available valences, up to two adjacent ring members may be a heteroatom, wherein one heteroatom is nitrogen and the other is selected from N, O or S. A heterocyclyl radical is derived by the removal of one hydrogen atom from a single carbon or nitrogen ring atom. A heterocyclyl linking group is derived by the removal of two hydrogen atoms each from either carbon or nitrogen ring atoms.

Heterocyclyl typically includes, but is not limited to, furyl, thienyl, 2H-pyrrole, 2-pyrrolinyl, 3-pyrrolinyl, pyrrolidinyl, pyrrolyl, 1,3-dioxolanyl, oxazolyl, thiazolyl, imidazolyl, 2-imidazolinyl (also referred to as 4,5-dihydro-1H-imidazolyl), imidazolidinyl, 2-pyrazolinyl, pyrazolidinyl, pyrazolyl, isoxazolyl, isothiazolyl, oxadiazolyl, triazolyl, thiadiazolyl, tetrazolyl, 2H-pyran, 4H-pyran, pyridinyl, piperidinyl, 1,4-dioxanyl, morpholinyl, 1,4-dithianyl, thiomorpholinyl, pyridazinyl, pyrimidinyl, pyrazinyl, piperazinyl, azepanyl, indolizinyl, indolyl, isoindolyl, 3H-indolyl, indolinyl, benzo[b]furyl, benzo[b]thienyl, 1H-indazolyl, benzimidazolyl, benzthiazolyl, purinyl, 4H-quinolizinyl, quinolinyl, isoquinolinyl, cinnolinyl, phthalzinyl, quinazolinyl, quinoxalinyl, 1,8-naphthyridinyl, pteridinyl, quinuclidinyl, hexahydro-1,4-diazepinyl, 1,3-benzodioxolyl (also known as 1,3-methylenedioxyphenyl), 2,3-dihydro-1,4-benzodioxinyl (also known as 1,4-ethylenedioxyphenyl), benzo-dihydro-furyl, benzo-tetrahydro-pyranyl, benzo-dihydro-thienyl, 5,6,7,8-tetrahydro-4H-cyclohepta(b)thienyl, 5,6,7-trihydro-4H-cyclohexa(b)thienyl, 5,6-dihydro-4H-cyclopenta(b)thienyl, hexahydro-cyclopenta[c]pyrrolyl, 2-aza-bicyclo[2.2.1]heptyl, 1-aza-bicyclo[2.2.2]octyl, 8-aza-bicyclo[3.2.1]octyl, 7-oxa-bicyclo[2.2.1]heptyl and the like.

The term “aryl” means an unsaturated, conjugated π electron monocyclic or polycyclic hydrocarbon ring system radical or linking group of 6, 9, 10 or 14 carbon atoms. An aryl radical is derived by the removal of one hydrogen atom from a single carbon ring atom. An arylene linking group is derived by the removal of two hydrogen atoms each of two carbon ring atoms. Aryl typically includes, but is not limited to, phenyl, naphthalenyl, azulenyl, anthracenyl and the like.

The term “alkylsulfonylamino” means a linking group of the formula -alkyl-SO₂NH—.

The term “alkylcarbamoyl” means a linking group of the formula -alkyl-C(O)NH—.

The term “amino” means a radical of the formula —NH₂ or a linking group of the formula —NH—.

The term “aminosulfonyl” means a radical of the formula —SO₂NH₂.

The term “arylalkoxy” means a radical of the formula —O-alkyl-aryl.

The term “aryloxy” means a radical of the formula —O-aryl.

The term “carbamoyl” means a radical of the formula —C(O)NH₂.

The term “carbamoylalkyl” means a radical of the formula —C(O)NH-alkyl or —C(O)N(alkyl)₂.

The term “carbonylalkoxy” means a radical of the formula —C(O)O-alkyl.

The term “carboxy” means a radical of the formula —COOH or —CO₂H.

The term “halo” or “halogen” means fluoro, chloro, bromo or iodo.

The term “lower alkyl-amino” means a radical of the formula —NH-alkyl or —N(alkyl)₂.

The term “lower alkyl-aminosulfonyl” means a radical of the formula —SO₂NH-alkyl or —SO₂N(alkyl)₂.

The term “lower alkyl-sulfonyl” means a radical of the formula —SO₂-alkyl or —C(O)N(alkyl)₂.

The substituent nomenclature used in the disclosure of the present invention was derived using nomenclature rules well known to those skilled in the art (e.g., IUPAC).

### Pharmaceutical Forms

The compounds of the present invention may be present in the form of pharmaceutically acceptable salts. For use in medicines, the “pharmaceutically acceptable salts” of the compounds of this invention refer to non-toxic acidic/anionic or basic/cationic salt forms.

Suitable pharmaceutically acceptable salts of the compounds of this invention include acid addition salts which may, for example, be formed by mixing a solution of the compound according to the invention with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid.

Furthermore when the compounds of the present invention carry an acidic moiety, suitable pharmaceutically acceptable salts thereof may include alkali metal salts, e.g. sodium or potassium salts; alkaline earth metal salts, e.g. calcium or magnesium salts; and salts formed with suitable organic ligands, e.g. quaternary ammonium salts. Thus, representative pharmaceutically acceptable salts include the following: acetate, benzenesulfonate, benzoate, bicarbonate, bisulfate, bitartrate, borate, bromide, calcium, camsylate (or camphorsulphonate), carbonate, chloride, clavulanate, citrate, dihydrochloride, edetate, fumarate, gluconate, glutamate, hydrabamine, hydrobromine, hydrochloride, iodide, isothionate, lactate, malate, maleate, mandelate, mesylate, nitrate, oleate, pamoate, palmitate, phosphate/diphosphate, salicylate, stearate, sulfate, succinate, tartrate, tosylate.

The present invention includes within its scope prodrugs and metabolites of the compounds of this invention. In general, such prodrugs and metabolites will be functional derivatives of the compounds that are readily convertible in vivo into an active compound.

The term “prodrug” means a pharmaceutically acceptable form of a functional derivative of a compound of the invention (or a salt thereof), wherein the prodrug may be: 1) a relatively active precursor which converts in vivo to an active prodrug component; 2) a relatively inactive precursor which converts in vivo to an active prodrug component; or 3) a relatively less active component of the compound that contributes to therapeutic biological activity after becoming available in vivo (i.e., as a metabolite). Conventional procedures for the selection and preparation of suitable prodrug derivatives are described in, for example, “Design of Prodrugs”, ed. H. Bundgaard, Elsevier, 1985.

The term “metabolite” means a pharmaceutically acceptable form of a metabolic derivative of a compound of the invention (or a salt thereof), wherein the derivative is a relatively less active component of the compound that contributes to therapeutic biological activity after becoming available in vivo.

The present invention contemplates compounds of various isomers and mixtures thereof. The term “isomer” refers to compounds that have the same composition and molecular weight but differ in physical and/or chemical properties. Such substances have the same number and kind of atoms but differ in structure. The structural difference may be in constitution (geometric isomers) or in an ability to rotate the plane of polarized light (stereoisomers).

The term “stereoisomer” refers to isomers of identical constitution that differ in the arrangement of their atoms in space. Enantiomers and diastereomers are stereoisomers wherein an asymmetrically substituted carbon atom acts as a chiral center. The term “chiral” refers to a molecule that is not superposable on its mirror image, implying the absence of an axis and a plane or center of symmetry. The term “enantiomer” refers to one of a pair of molecular species that are mirror images of each other and are not superposable. The term “diastereomer” refers to stereoisomers that are not related as mirror images. The symbols “R” and “S” represent the configuration of substituents around a chiral carbon atom(s). The symbols “R*” and “S*” denote the relative configurations of substituents around a chiral carbon atom(s).

The term “racemate” or “racemic mixture” refers to a compound of equimolar quantities of two enantiomeric species, wherein the compound is devoid of optical activity. The term “optical activity” refers to the degree to which a chiral molecule or nonracemic mixture of chiral molecules rotates the plane of polarized light.

The term “geometric isomer” refers to isomers that differ in the orientation of substituent atoms in relationship to a carbon-carbon double bond, to a cycloalkyl ring or to a bridged bicyclic system. Substituent atoms (other than H) on each side of a carbon-carbon double bond may be in an E or Z configuration. In the “E” (opposite sided) or “chair” configuration, the substituents are on opposite sides in relationship to the carbon-carbon double bond; in the “Z” (same sided) or “boat” configuration, the substituents are oriented on the same side in relationship to the carbon-carbon double bond. Substituent atoms (other than H) attached to a carbocyclic ring may be in a cis or trans configuration. In the “cis” configuration, the substituents are on the same side in relationship to the plane of the ring; in the “trans” configuration, the substituents are on opposite sides in relationship to the plane of the ring. Compounds having a mixture of “cis” and “trans” species are designated “cis/trans”. Substituent atoms (other than H) attached to a bridged bicyclic system may be in an “endo” or “exo” configuration. In the “endo” configuration, the substituents attached to a bridge (not a bridgehead) point toward the larger of the two remaining bridges; in the “exo” configuration, the substituents attached to a bridge point toward the smaller of the two remaining bridges.

It is to be understood that the various substituent stereoisomers, geometric isomers and mixtures thereof used to prepare compounds of the present invention are either commercially available, can be prepared synthetically from commercially available starting materials or can be prepared as isomeric mixtures and then obtained as resolved isomers using techniques well-known to those of ordinary skill in the art.

The isomeric descriptors “R,” “S,” “S*,” “R*,” “E,” “Z,” “cis,” “trans,” “exo” and “endo” are used as described herein for indicating atom configuration(s) relative to a core molecule and are intended to be used as defined in the literature (IUPAC Recommendations for Fundamental Stereochemistry (Section E), Pure Appl. Chem., 1976, 45:13-30).

Furthermore, compounds of the present invention may have one or more polymorph or amorphous crystalline forms and as such are intended to be included in the scope of the invention. In addition, some of the compounds may form solvates with water (i.e., hydrates) or common organic solvents, and such are also intended to be encompassed within the scope of this invention.

### Therapeutic Use

The CB2 receptor belongs to the G-protein-coupled receptor (GCPR) family and appears to be primarily expressed peripherally in lymphoid tissue (cell mediated and innate immunity), peripheral nerve terminals (peripheral nervous system), spleen immune cells (immune system modulation) and retina (intraocular pressure). CB2 mRNA is found in the CNS in cerebellar granule cells (coordinating motor function).

Activation of the CB2 receptor by an agonist compound mediates pain responses in animal models.

The present invention is directed to a method for treating, ameliorating or preventing CB2 receptor mediated pain in a subject in need thereof comprising administering to the subject an effective amount of a compound of formula (I) or formula (Ia) or a form thereof.

The term “CB2 receptor mediated pain” as used herein, refers to pain states that are chronic or acute, that are postoperative, inflammatory or neuropathic or the result of injury or age and include, without limitation, central and peripheral pathway mediated pain states that otherwise defy characterization and would benefit from treatment with a CB2 receptor agonist.

The scope of the present method is intended to include inflammatory related pain states selected from the group consisting of osteoarthritis, rheumatoid arthritis, headache, migraine, odontaligia, labor, dysmenorrhea, interstitial cystitis, peripheral neuritis, mucositis, surgery pain, sports injury pain, trauma, cancer pain, fibromyalgia, pancreatitis, enteritis, cellulitis, bony fractures, post-operative ileus, irritable bowel syndrome, pain due to inflammatory bowel diseases, Crohn's Disease, ulcerative colitis, cholecystitis, burn, sunburn, pain due to venomous snake bite, spider bite or insect sting and pain due to nonvenomous snake bite, spider bite or insect sting.

The scope of the present method is further intended to include neuropathic related pain states selected from the group consisting of chemotherapeutic neuropathy, AIDS-related neuropathy, diabetic neuropathy and post herpetic neuralgia.

An example of the present invention includes use of a compound of formula (I) or formula (Ia) or a form thereof in the manufacture of a medicament for treating, ameliorating or preventing CB2 receptor mediated pain in a subject in need thereof.

An example of the present invention includes a method for treating, ameliorating or preventing CB2 receptor mediated pain in a subject in need thereof comprising administering to the subject a combination product and/or therapy comprising an effective amount of a compound of formula (I) or formula (Ia) or a form thereof and a therapeutic agent.

Compounds of formula (I) or formula (Ia) are CB2 agonists useful in the method of the present in the invention, having a CB2 agonist binding activity IC₅₀ value of between about 50 μM to about 0.01 nM; between about 25 μM to about 0.01 nM; between about 15 μM to about 0.01 nM; between about 10 μM to about 0.01 nM; between about 1 μM to about 0.01 nM; between about 800 nM to about 0.01 nM; between about 200 nM to about 0.01 nM; between about 100 nM to about 0.01 nM; between about 80 nM to about 0.01 nM; between about 20 nM to about 0.01 nM; between about 10 nM to about 0.1 nM; or about 0.1 nM.

The term “subject” as used herein, refers to a patient, which may be an animal, preferably a mammal, most preferably a human, which has been the object of treatment, observation or experiment and is at risk of (or susceptible to) developing a CB receptor mediated syndrome, disorder or disease.

The term “administering” is to be interpreted in accordance with the methods of the present invention. Such methods include therapeutically or prophylactically administering an effective amount of a compound of formula (I) or formula (Ia) at different times during the course of a therapy or concurrently as a product in a combination form. Thus, in the methods of treatment of the present invention, the term shall encompass the means for treating, ameliorating or preventing the CB2 receptor mediated pain described herein with a compound specifically disclosed or a prodrug or metabolite thereof, which would obviously be included within the scope of the invention albeit not specifically disclosed for certain of the instant compounds.

Prophylactic administration can occur prior to the manifestation of symptoms characteristic of CB2 receptor mediated pain such that the pain is treated, ameliorated, prevented or otherwise delayed in its progression. The methods of the present invention are further to be understood as embracing all therapeutic or prophylactic treatment regimens used by those skilled in the art.

The term “effective amount” refers to that amount of an instant compound that elicits the biological or medicinal response in a tissue system, animal or human, that is being sought by a researcher, veterinarian, medical doctor, or other clinician, which includes alleviation of the symptoms of the syndrome, disorder or disease being treated. The effective amount of such a compound for use in the present invention is from about 0.001 mg/kg/day to about 300 mg/kg/day.

The term “medicament” refers to a product for use in treating, ameliorating or preventing a cannabinoid receptor mediated syndrome, disorder or disease.

The term “combination product and/or therapy” means a pharmaceutical composition comprising a compound of formula (I) or formula (Ia) in combination with one or more therapeutic agents. The dosages of the compound of formula (I) or formula (Ia) and the one or more therapeutic agents are adjusted when combined to achieve an effective amount.

Wherein the present invention is directed to the administration of a combination product, the term “effective amount” means that amount of the combination of agents taken together so that the combined effect elicits the desired biological or medicinal response.

As those skilled in the art will appreciate, the effective amounts of the components comprising the combination product may be independently optimized and combined to achieve a synergistic result whereby the pathology is reduced more than it would be if the components of the combination product were used alone.

Wherein the present invention is directed to the administration of a combination product and/or therapy, an instant compound and the agent may be co-administered by any suitable means, simultaneously, sequentially, alternately or in a single or divided form, at the same or different times during the course of therapy.

Where an instant compound and the agent components are administered separately, the number of dosages of an instant compound given per day, may not necessarily be the same, e.g. where one compound may have a greater duration of activity, and will therefore, be administered less frequently.

Suitable examples of methods of administration are orally, intravenous (iv), intramuscular (im), subcutaneous (sc), transdermal and topical. Compounds may also be administrated directly to the nervous system including, but not limited to the intracerebral, intraventricular, intracerebroventricular, intrathecal, intracisternal, intraspinal and/or peri-spinal routes of administration by delivery via intracranial or intravertebral needles and/or catheters with or without pump devices.

Optimal dosages to be administered may be readily determined by those skilled in the art, and will vary with the particular compound used, the mode of administration, the strength of the preparation and the advancement of the disease condition. In addition, factors associated with the particular patient being treated, including patient's sex, age, weight, diet, time of administration and concomitant diseases, will result in the need to adjust dosages.

The present invention includes administration of a pharmaceutical composition or medicament comprising an admixture of a compound of the present invention and an optional pharmaceutically acceptable carrier.

### Pharmaceutical Compositions

The term “composition” refers to a product comprising the specified ingredients in the specified amounts, as well as any product that results, directly or indirectly, from combinations of the specified ingredients in the specified amounts.

Pharmaceutical compositions of the invention may, alternatively or in addition to a compound of formula (I) or formula (Ia), comprise a pharmaceutically acceptable salt of a compound of formula (I) or formula (Ia) or a prodrug or pharmaceutically active metabolite of such a compound or salt in admixture with a pharmaceutically acceptable carrier.

“Pharmaceutically acceptable carrier” means molecular entities and compositions that are of sufficient purity and quality for use in the formulation of a composition of the invention and that, when appropriately administered to an animal or a human, do not produce an adverse, allergic, or other untoward reaction.

Since both clinical and veterinary uses are equally included within the scope of the present invention, a pharmaceutically acceptable formulation would include a composition or medicament formulation for either clinical or veterinary use.

The composition or medicament may be administered in a wide variety of dosage unit forms depending on the method of administration; wherein such methods include (without limitation) oral, sublingual, nasal (inhaled or insufflated), transdermal, rectal, vaginal, topical (with or without occlusion), intravenous (bolus or infusion) or for injection (intraperitoneally, subcutaneously, intramuscularly, intratumorally or parenterally) using a suitable dosage form well known to those of ordinary skill in the area of pharmaceutical administration. Accordingly, the term “dosage unit” or “dosage form” is alternatively used to refer to (without limitation) a tablet, pill, capsule, solution, syrup, elixir, emulsion, suspension, suppository, powder, granule or sterile solution, emulsion or suspension (for injection from an ampoule or using a device such as an auto-injector or for use as an aerosol, spray or drop). Furthermore, the composition may be provided in a form suitable for weekly or monthly administration (e.g. as an insoluble salt of the active compound (such as the decanoate salt) adapted to provide a depot preparation for intramuscular injection).

The present invention includes a composition of an instant compound or prodrug thereof present in a prophylactically or therapeutically effective amount necessary for symptomatic relief to a subject in need thereof A prophylactically or therapeutically effective amount of an instant compound or prodrug thereof may range from about 0.001 mg to about 1 g and may be constituted into any form suitable for the administration method and regimen selected for the subject.

Depending on the subject and disease to be treated, the prophylactically or therapeutically effective amount for a person of average body weight of about 70 kg per day may range from about 0.001 mg/kg to about 300 mg/kg; from about 0.01 mg/kg to about 200 mg/kg; from about 0.05 mg/kg to about 100 mg/kg; or, from about 0.1 mg/kg to about 50 mg/kg.

An optimal prophylactically or therapeutically effective amount and administration method and regimen may be readily determined by those skilled in the art, and will vary depending on factors associated with the particular patient being treated (age, weight, diet and time of administration), the severity of the condition being treated, the compound and dosage unit being employed, the mode of administration and the strength of the preparation.

Dosage unit(s) may be administered to achieve the therapeutically or prophylactically effective amount in a regimen of from about once per day to about 5 times per day. The preferred dosage unit for oral administration is a tablet containing 0.01, 0.05, 0.1, 0.5, 1.0, 2.5, 5.0, 10.0, 15.0, 25.0, 50.0, 100, 150, 200, 250 or 500 mg of the active ingredient.

### BIOLOGICAL EXAMPLES

The following examples illustrate that the compounds of the present invention are useful in a method for treating, ameliorating or preventing CB2 receptor mediated pain in a subject in need thereof.

### Example 1

### Carrageenan Model of Inflammatory Pain

Intraplantar injection of carrageenan (Cg) in a rodent produces pronounced hypersensitivity to both thermal and mechanical stimuli. The effects of carrageenan are maximal 2-4 hr after administration.

### Procedure

To assess the ability of test compounds to reverse thermal hyperalgesia, baseline response latencies on a radiant heat (RH) paw stimulator were obtained before an intraplantar injection of carrageenan-λ (200 μL) in male Sprague-Dawley rats (250-350 g in treatment groups of 9 animals each). Only withdrawal responses that were quick hind paw movements (with or without licking of the hind paw) were recorded. Paw movements associated with locomotion or a shifting of weight were not considered a withdrawal response.

The weight of each animal was recorded on the day of the experiment. Each animal was placed on a warm (approx. body temperature, 30° C.) glass surface and allowed to acclimate to the test chamber for approximately 10-15 minutes. A radiant thermal stimulus (beam of light) was then focused on the sole of each hind paw in turn, and an initial (baseline) response time to thermal stimuli was recorded for each animal. The stimulus intensity (radiant heat at a setting of 5 Amps) that produced 10-15 sec baseline withdrawal latencies was used and a maximum cutoff of 20 sec was imposed. The light stimulus was automatically shut off by a photoelectric relay when the foot moved or when the cut-off time limit was reached.

One treatment group (8 animals each) was injected i.p. with vehicle (5% DMSO and 5% Tween-80 in sterile saline). The other treatment groups (8 animals each) were injected i.p. with 3, 10 or 30 mg/kg Compound 7.

One hour later, withdrawal latencies for the animals administered vehicle were recorded. After assessment, all animals were administered 1% carrageenan (200 μL in sterile saline) subcutaneously into the sub-plantar tissue of the left hind paw to stimulate an acute inflammatory reaction. Three hours later, the response time of the animals to the thermal stimulus was evaluated. The results are shown in Table 1 as seconds ±SEM.

TABLE 1

| | | 1 hr post | |
|---|---|---|---|
| | | vehicle | 3 hrs post Cg |
| | Baseline | administration | administration |
| Vehicle | 13.14 ± 0.85 | 12.43 ± 1.50 | 7.21 ± 1.87 |
| 3 mg/kg | | | 8.44 ± 1.88 |
| 10 mg/kg | | | 7.87 ± 1.01 |
| 30 mg/kg | | | 6.05 ± 0.83 |

Three hours after carrageenan (Cg) administration, mean latencies in vehicle treated animals were significantly decreased, indicating the development of thermal hyperalgesia.

### Example 2

The experiment of Example 1 was repeated with the exception that animals were first administered 1% carrageenan (200 μL in sterile saline) subcutaneously into the sub-plantar tissue of the left hind paw to stimulate an acute inflammatory reaction.

Two and a half hours later, withdrawal latencies were assessed (‘post-Cg’). One treatment group (7 animals each) was then injected i.p. with vehicle (5% DMSO and 5% Tween-80 in sterile saline). The other treatment groups (8 animals each) were injected i.p. with 3, 10 or 30 mg/kg Compound 7. Thirty minutes after test compound administration withdrawal latencies were recorded. The results are shown in Table 2 as seconds ±SEM.

TABLE 2

| | Baseline | Post Cg | 0.5 hrs post Cg |
|---|---|---|---|
| Vehicle | 12.72 ± 0.59 | 7.12 ± 0.46 | 4.32 ± 0.53 |
| 3 mg/kg | 12.51 ± 0.74 | 7.45 ± 0.43 | 5.30 ± 0.48 |
| 10 mg/kg | 12.14 ± 0.52 | 7.43 ± 0.55 | 5.33 ± 0.48 |
| 30 mg/kg | 12.79 ± 0.55 | 7.00 ± 0.39 | 4.53 ± 0.39 |

After carrageenan (Cg) administration, mean latencies in vehicle treated animals were significantly decreased, indicating the development of thermal hypersensitivity.

### Example 3

### Hot-Plate Nociception Test

The hot-plate test originally described by Eddy and Leimbach (J. Pharmacol. Exp. Ther. 107:385-393, 1953) with minor modifications (e.g., O'Callaghan and Holtzman, J. Pharmacol. Exp. Ther. 192: 497-505, 1975) was used to ascertain the analgesic potential of investigated compounds. The hot plate analgesia meter used for these studies was produced by Columbus Instruments International (Columbus, Ohio).

### Procedure

Male CD-1 mice (30-35 g) were weighed, placed in a plastic box with wood chips and allowed to acclimate before testing. An individual mouse was placed on a 48° C. heated surface and locomotion on the plate was constrained by a glass cylinder. The time interval between placement and a shaking, licking or tucking of either hind-paw (nociceptive response) was recorded as the Baseline measurement. Animals were removed from the heated plate immediately after responding or after a maximum of 40 sec to prevent tissue damage. Each mouse was tested only once.

One treatment group (9 animals each) was then injected i.p. with vehicle (5% DMSO and 5% Tween-80 in sterile saline). The other treatment groups (8 animals each) were injected i.p. with 10 or 30 mg/kg Compound I. Thirty minutes after test compound administration, each animal was assessed for a response with a maximum cut-off of 90 sec.

The reaction time for a vehicle or test compound treated animal was compared to the respective baseline reaction time corresponding to each animal. The percent maximal effect (% MPE) was obtained by subtracting the baseline response time from the post-treatment response time and dividing the result by the difference of the baseline response time subtracted from the cut-off time (90 sec). The results are shown in Table 3 as % MPE ±SEM.

TABLE 3

| | % MPE Vehicle | % MPE |
|---|---|---|
| 10 mg/kg | 15.51 ± 15.06 | 20.58 ± 9.27 |
| 30 mg/kg | 6.32 ± 13.06 | 4.25 ± 10.65 |

### Example 4

### Visceral Hyperalgesia Model

This protocol uses barostat-controlled, isobaric colorectal distensions (CRD) in rats to evaluate the potency and efficacy of test compounds in treating visceral hyperalgesia.

### Procedure

Rats (male Sprague Dawley (275-350 g; CD(SD); Charles River Labs) are housed 2 to 4 animals per cage in a temperature and humidity controlled room with a 12 hr/12 hr light/dark cycle, with ad libitum access to food and water.

One day after release from quarantine, the animals were acclimated to progressively longer (30 min and 4 hr later, 45 min) periods of simple restraint in plexiglas devices (G-3, rat ECU; Braintree Scientific; Braintree Mass.). The animals were returned to their home cages overnight. The next day they were acclimated in the restraint device for 60 min in the morning. 4 hrs later, the animals were lightly anesthetized with 70% CO₂:30% O₂. A highly compliant, 4 cm long polyethylene balloon, lubricated with K-Y Jelly was then inserted via the anus into the rectum and distal colon. The balloon was positioned such that the aboral end was 1 cm from the anus and was secured in place by taping the balloon catheter to the base of the tail. The catheter was connected to a computerized barostat that controlled the inflation of the balloon and the resulting colorectal distension. The balloon pressure, representing intracolonic pressure, was continuously recorded.

CRD in conscious animals elicits a reflex visceromotor response consisting of contraction of the anterior abdominal wall muscles (Ness T J and Gebhart G F, Colorectal distension as a noxious visceral stimulus: physiologic and pharmacologic characterization of pseudaffective reflexes in the rat, Brain Res., (1988), 450: 153-169). Contraction of these muscles increases intraabdominal pressure and subsequently increases intracolonic pressure. Changes in intracolonic pressure are transduced through the same balloon used to deliver the CRD. The manometric endpoint has recently been reported to mimic electromyographic responses recorded from anterior abdominal wall muscles in rats (Tammpere A, Brusberg M, Axenborg J, Hirsch I, Larsson H and Lindstrom E, Evaluation of pseudo-affective responses to noxious colorectal distension in rats by manometric recordings, Pain, (2005), 116: 220-226)

Stimulus-response data were obtained by delivering two series of 20 sec ramp (15, 30, 45, 60, 75 mmHg) distensions at four-minute intervals and recording the manometric response as follows: the intracolonic pressure signal is passed through a digital 1 Hz highpass filter, rectified and the integral of the initial 15 seconds of the CRD subjected to baseline subtraction (the 15 sec immediately preceding balloon distension); the responses at each distending pressure are averaged to obtain a control stimulus/response curve for each animal. The colorectal balloons were then removed and the animals were returned to their home cages.

The following morning, one treatment group (4 animals) was injected i.p. with 10 mg/kg Compound 7 (solubilized in 5% DMSO and 5% Tween-80 in sterile saline).

One hour later, an acute colitis was induced in all treatment groups by the intracolonic instillation of a 1.5 mL bolus of 2.5% (w/v) zymosan A (from Saccharomyces cerevisiae; Sigma Chemical Co., St. Louis) in 30% ethanol (under light 70% CO₂:30% O₂ anesthesia). Four hours later, the animals were lightly anesthetized and the colorectal balloons inserted as on the previous day for controlled distensions. The identical CRD stimuli was applied and manometric responses were recorded and analyzed as described for the control phase of the experiment.

The animals in one treatment group (4 animals) were then subcutaneously (s.c.) dosed with 1 mg/kg morphine. As a comparator for analgesic response, animals in another treatment group (9 animals) were dosed s.c. with 3 mg/kg morphine 4 hrs after colitis initiation and 30 min prior to CRD. Data were excluded from experiments in which animals in the vehicle treatment group (6 animals) did not exhibit a hyperalgesic response following zymosan administration. Data are expressed in Table 4 as a percent (% ±SEM) of the initial (control) manometric responses, with each animal serving as its own control.

TABLE 4

| | | 3 mpk Morphine | Compound 7 | Compound 7/1 mpk |
|---|---|---|---|---|
| | Vehicle Treatment | Treatment | Treatment | Morphine Treatment |
| mmHg | Group (n = 6) | Group (n = 9) | Group (n = 4) | Group (n = 4) |
| 15 | 135.5 ± 39.72 | 96.34 ± 12.53 | 245.7 ± 87.76 | 124.1 ± 24.83 |
| 30 | 277.0 ± 76.31 | 69.96 ± 11.21 | 527.2 ± 196.8 | 164.5 ± 89.14 |
| 45 | 383.8 ± 104.9 | 61.69 ± 8.72 | 301.3 ± 104.2 | 141.7 ± 32.83 |
| 60 | 236.6 ± 53.28 | 56.33 ± 8.23 | 243.7 ± 66.53 | 133.6 ± 24.98 |
| 75 | 166.0 ± 32.01 | 63.18 ± 9.19 | 230.9 ± 51.95 | 171.5 ± 29.46 |

It is to be understood that the preceding description of the invention and various examples thereof have emphasized certain aspects. Numerous other equivalents not specifically elaborated on or discussed may nevertheless fall within the spirit and scope of the present invention or the following claims and are intended to be included.

What is claimed is:

1. A method for treating, ameliorating or preventing CB2 receptor mediated pain in a subject in need thereof comprising administering to the subject an effective amount of a compound of formula (I): 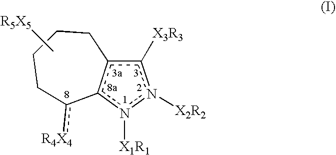
or a form thereof, wherein
the dashed lines between positions 2-3 and positions 3a-8a in formula (I) represent locations for each of two double bonds present when X₁R₁ is present;
the dashed lines between positions 3-3a and positions 8a-1 in formula (I) represent locations for each of two double bonds present when X₂R₂ is present;
the dashed line between position 8 and X₄R₄ in formula (I) represents the location for a double bond;
X₁ is absent or lower alkylene;
X₂ is absent or lower alkylene;
wherein only one of X₁R₁ and X₂R₂ are present;
X₃ is absent, lower alkylene, lower alkylidene or —NH—;
when the dashed line between position 8 and X₄R₄ is absent, X₄ is absent, or is lower alkylene;
when the dashed line between position 8 and X₄R₄ is present, X₄ is absent;
X₅ is absent or lower alkylene;
R₁ is selected from hydrogen, alkyl (optionally substituted at one or more positions by halogen, hydroxy or lower alkoxy), lower alkyl-sulfonyl, aryl, C₃-C₁₂ cycloalkyl or heterocyclyl, wherein aryl, C₃-C₁₂ cycloalkyl or heterocyclyl are each optionally substituted at one or more positions by halogen, aminosulfonyl, lower alkyl-aminosulfonyl, alkyl (optionally substituted at one or more positions by halogen, hydroxy or lower alkoxy), hydroxy or lower alkoxy (optionally substituted at one or more positions by halogen or hydroxy);
R₂ is selected from hydrogen, alkyl (optionally substituted at one or more positions by halogen, hydroxy or lower alkoxy), lower alkyl-sulfonyl, aryl, C₃-C₁₂ cycloalkyl or heterocyclyl, wherein aryl, C₃-C₁₂ cycloalkyl or heterocyclyl are each optionally substituted at one or more positions by halogen, aminosulfonyl, lower alkyl-aminosulfonyl, alkyl (optionally substituted at one or more positions by halogen, hydroxy or lower alkoxy), hydroxy or lower alkoxy (optionally substituted at one or more positions by halogen or hydroxy);
R₃ is —C(O)-Z₁(R₆), —SO₂—NR₇-Z₂(R₅) or —C(O)—NR₉-Z₃(R₁₀);
when the dashed line between position 8 and X₄R₄ is absent, X₄ is absent or lower alkylene and R₄ is hydroxy, lower alkoxy, halogen, aryl, C₃-C₁₂ cycloalkyl or heterocyclyl, wherein aryl, C₃-C₁₂ cycloalkyl or heterocyclyl are each optionally substituted at one or more positions by hydroxy, oxo, lower alkyl (optionally substituted at one or more positions by halogen, hydroxy or lower alkoxy), lower alkoxy (optionally substituted at one or more positions by halogen or hydroxy) or halogen;
when the dashed line between position 8 and X₄R₄ is present, X₄ is absent and
R₄ is CH-aryl or CH-heterocyclyl, wherein aryl or heterocyclyl are each optionally substituted at one or more positions by hydroxy, oxo, lower alkyl (optionally substituted at one or more positions by halogen, hydroxy or lower alkoxy), lower alkoxy (optionally substituted at one or more positions by halogen or hydroxy) or halogen;
R₅ is hydrogen, hydroxy, oxo, halogen, amino, lower alkyl-amino, alkyl (optionally substituted at one or more positions by halogen, hydroxy or lower alkoxy), lower alkoxy (optionally substituted at one or more positions by halogen or hydroxy), carboxy, carbonylalkoxy, carbamoyl, carbamoylalkyl, aryl, aryloxy, arylalkoxy or heterocyclyl;
R₆ is aryl, C₃-C₁₂ cycloalkyl or heterocyclyl each optionally substituted by one or more hydroxy, oxo, halogen, amino, lower alkyl-amino, alkyl (optionally substituted at one or more positions by halogen, hydroxy or lower alkoxy), lower alkoxy (optionally substituted at one or more positions by halogen or hydroxy), carboxy, carbonylalkoxy, carbamoyl, carbamoylalkyl, aryl, aryloxy, arylalkoxy or heterocyclyl;
R₇ is hydrogen or lower alkyl;
R₈ is hydrogen, aryl, C₃-C₁₂ cycloalkyl or heterocyclyl, wherein aryl, C₃-C₁₂ cycloalkyl or heterocyclyl are each optionally substituted by one or more hydroxy, oxo, halogen, amino, lower alkyl-amino, alkyl (optionally substituted at one or more positions by halogen, hydroxy or lower alkoxy), lower alkoxy (optionally substituted at one or more positions by halogen or hydroxy), carboxy, carbonylalkoxy, carbamoyl, carbamoylalkyl, aryl, aryloxy, arylalkoxy or heterocyclyl;
R₉ is hydrogen or lower alkyl;
R₁₀ is hydrogen, aryl, C₃-C₁₂ cycloalkyl or heterocyclyl, wherein aryl, C₃-C₁₂ cycloalkyl or heterocyclyl are each optionally substituted by one or more hydroxy, oxo, halogen, amino, lower alkyl-amino, alkyl (optionally substituted at one or more positions by halogen, hydroxy or lower alkoxy), lower alkoxy (optionally substituted at one or more positions by halogen or hydroxy), carboxy, carbonylalkoxy, carbamoyl, carbamoylalkyl, aminosulfonyl, lower alkyl-aminosulfonyl, aryl, aryloxy, arylalkoxy or heterocyclyl;
Z₁ and Z₂ are each absent or alkyl; and,
Z₃ is absent, —NH—, —SO₂— or alkyl (wherein alkyl is optionally substituted at one or more positions by halogen, hydroxy, lower alkyl, lower alkoxy, carboxy or carbonylalkoxy).

2. The method of claim 1, wherein X₁ is absent and R₁ is selected from hydrogen, alkyl, lower alkyl-sulfonyl, aryl, C₃-C₁₂ cycloalkyl or heterocyclyl, wherein aryl or heterocyclyl are each optionally substituted at one or more positions by halogen, aminosulfonyl or alkyl (optionally substituted at one or more positions by halogen).

3. The method of claim 1, wherein R₃ is —SO₂—NR₇-Z₂(R₅); X₃ is absent or lower alkylidene; R₇ is hydrogen or lower alkyl; Z₂ is absent or alkyl; and, R₈ is aryl, C₃-C₁₂ cycloalkyl or heterocyclyl.

4. The method of claim 1, wherein R₃ is —SO₂—NH-Z₂(R₅); X₃ is absent or lower alkylidene; Z₂ is absent or alkyl; and, R₈ is aryl, C₃-C₁₂ cycloalkyl or heterocyclyl.

5. The method of claim 1, wherein R₃ is —C(O)—NR₉-Z₃(R₁₀); X₃ is absent or lower alkylidene; R₉ is hydrogen or lower alkyl; Z₃ is absent, —SO₂— or alkyl (wherein alkyl is optionally substituted at one or more positions by halogen, hydroxy or carbonylalkoxy); and, R₁₀ is hydrogen, aryl, C₃-C₁₂ cycloalkyl or heterocyclyl, wherein aryl, C₃-C₁₂ cycloalkyl or heterocyclyl are each optionally substituted by one or more hydroxy, halogen, alkyl (optionally substituted at one or more positions by halogen), alkoxy, carboxy, carbonylalkoxy, carbamoylalkyl or aminosulfonyl.

6. The method of claim 1, wherein R₃ is —C(O)—NH-Z₃(R₁₀); X₃ is absent or lower alkylidene; Z₃ is absent, —SO₂— or alkyl (wherein alkyl is optionally substituted at one or more positions by halogen, hydroxy or carbonylalkoxy); and, R₁₀ is hydrogen, aryl, C₃-C₁₂ cycloalkyl or heterocyclyl, wherein aryl, C₃-C₁₂ cycloalkyl or heterocyclyl are each optionally substituted by one or more hydroxy, halogen, alkyl (optionally substituted at one or more positions by halogen), alkoxy, carboxy, carbonylalkoxy, carbamoylalkyl or aminosulfonyl.

7. The method of claim 1, wherein R₃ is —C(O)—NH-Z₃(R₁₀); X₃ is absent or lower alkylidene; Z₃ is absent, —SO₂— or alkyl (wherein alkyl is optionally substituted at one or more positions by halogen, hydroxy or carbonylalkoxy); and, R₁₀ is aryl optionally substituted by one or more hydroxy, halogen, alkyl (optionally substituted at one or more positions by halogen), alkoxy or aminosulfonyl.

8. The method of claim 1, wherein R₃ is —C(O)—NH-Z₃(R₁₀); X₃ is absent or lower alkylidene; Z₃ is absent, —SO₂— or alkyl (wherein alkyl is optionally substituted at one or more positions by halogen, hydroxy or carbonylalkoxy); and, R₁₀ is hydrogen or C₃-C₁₂ cycloalkyl, wherein C₃-C₁₂ cycloalkyl is optionally substituted by one or more hydroxy, alkyl, alkoxy, carboxy, carbonylalkoxy or carbamoylalkyl.

9. The method of claim 1, wherein R₃ is —C(O)—NH-Z₃(R₁₀); X₃ is absent or lower alkylidene; Z₃ is absent, —SO₂— or alkyl (wherein alkyl is optionally substituted at one or more positions by halogen, hydroxy or carbonylalkoxy); and, R₁₀ is hydrogen or heterocyclyl, wherein heterocyclyl is optionally substituted by one or more carbonylalkoxy.

10. The method of claim 1, wherein the dashed line between position 8 and X₄R₄ is absent, X₄ is absent or lower alkylene and R₄ is aryl optionally substituted at one or more positions by lower alkyl or halogen.

11. The method of claim 1, wherein the dashed line between position 8 and X₄R₄ is present, X₄ is absent and R₄ is CH-aryl or CH-heterocyclyl, wherein aryl or heterocyclyl are each optionally substituted at one or more positions by lower alkoxy or halogen.

12. The method of claim 1, wherein X₅ is absent and R₅ is hydrogen.

13. The method of claim 1, wherein the compound is selected from a compound of formula (Ia) 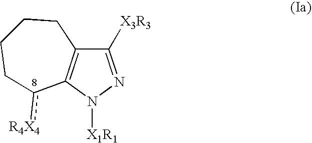
or a salt, isomer, prodrug, metabolite or polymorph thereof wherein X₁ is absent or lower alkylene; X₃ is absent or lower alkylidene; X₄ is absent or is lower alkylene when the dashed line between position 8 and X₄R₄ is absent; X₄ is absent when the dashed line between position 8 and X₄R₄ is present; R₁ is selected from hydrogen, alkyl, lower alkyl-sulfonyl, aryl, C₃-C₁₂ cycloalkyl or heterocyclyl, wherein aryl or heterocyclyl are each optionally substituted at one or more positions by halogen, aminosulfonyl or alkyl (optionally substituted at one or more positions by halogen); R₃ is —C(O)—(R₆), —SO₂—NH-Z₂(R₈) or —C(O)—NH-Z₃(R₁₀); when the dashed line between position 8 and X₄R₄ is absent, R₄ is aryl, wherein aryl is optionally substituted at one or more positions by lower alkyl or halogen; when the dashed line between position 8 and X₄R₄ is present, R₄ is CH-aryl or CH-heterocyclyl, wherein aryl or heterocyclyl are each optionally substituted at one or more positions by lower alkoxy or halogen; R₆ is heterocyclyl optionally substituted by one or more aryl or heterocyclyl; Z₂ is absent or alkyl; R₈ is aryl, C₃-C₁₂ cycloalkyl or heterocyclyl; Z₃ is absent, —SO₂— or alkyl (wherein alkyl is optionally substituted at one or more positions by halogen, hydroxy or carbonylalkoxy); and, R₁₀ is hydrogen, aryl, C₃-C₁₂ cycloalkyl or heterocyclyl, wherein aryl, C₃-C₁₂ cycloalkyl or heterocyclyl are each optionally substituted by one or more hydroxy, halogen, alkyl (optionally substituted at one or more positions by halogen), alkoxy, carboxy, carbonylalkoxy, carbamoylalkyl or aminosulfonyl.

14. The method of claim 1, wherein X₁ is absent; X₃ is absent or lower alkylidene; X₄ is lower alkylene when the dashed line between position 8 and X₄R₄ is absent; X₄ is absent when the dashed line between position 8 and X₄R₄ is present; R₁ is selected from hydrogen or alkyl; R₃ is —SO₂—NH-Z₂(R₈) or —C(O)—NH-Z₃(R₁₀); when the dashed line between position 8 and X₄R₄ is absent, R₄ is aryl, wherein aryl is optionally substituted at one or more positions by lower alkyl or halogen; when the dashed line between position 8 and X₄R₄ is present, R₄ is CH-aryl or CH-heterocyclyl, wherein aryl or heterocyclyl are each optionally substituted at one or more positions by lower alkoxy or halogen; Z₂ is absent or alkyl; R₈ is aryl or heterocyclyl; Z₃ is alkyl (wherein alkyl is optionally substituted at one or more positions by halogen, hydroxy or carbonylalkoxy); and, R₁₀ is aryl or heterocyclyl, wherein aryl or heterocyclyl are each optionally substituted by one or more hydroxy, halogen, alkyl (optionally substituted at one or more positions by halogen), alkoxy, carboxy, carbonylalkoxy, carbamoylalkyl or aminosulfonyl.

15. The method of claim 1, wherein the compound is selected from:
8-(3-chloro-benzyl)-1,4,5,6,7,8-hexahydro-cycloheptapyrazole-3-carboxylic acid [(1S)-2-hydroxy-1-phenyl-ethyl]-amide,
(8R*)-8-(3-chloro-benzyl)-1,4,5,6,7,8-hexahydro-cycloheptapyrazole-3-carboxylic acid [(1R)-2-hydroxy-1-phenyl-ethyl]-amide,
(8R*)-8-(3-chloro-benzyl)-1,4,5,6,7,8-hexahydro-cycloheptapyrazole-3-carboxylic acid [(S)-2-hydroxy-1-phenyl-ethyl]-amide,
(8S*)-8-(3-chloro-benzyl)-1,4,5,6,7,8-hexahydro-cycloheptapyrazole-3-carboxylic acid [(S)-2-hydroxy-1-phenyl-ethyl]-amide,
(2E)-2-[(8R*)-8-(3-fluoro-benzyl)-1,4,5,6,7,8-hexahydro-cycloheptapyrazol-3-yl]-ethenesulfonic acid [(S)-1-phenyl-ethyl]-amide,
(8E)-8-(4-chloro-benzylidene)-1,4,5,6,7,8-hexahydro-cycloheptapyrazole-3-carboxylic acid [(1R)-2-hydroxy-1-phenyl-ethyl]-amide,
(2E,8E)-2-[8-(4-chloro-benzylidene)-1,4,5,6,7,8-hexahydro-cycloheptapyrazol-3-yl]-ethenesulfonic acid [(1S)-1-phenyl-ethyl]-amide,
(8E)-(2S)-2-{[8-(4-chloro-benzylidene)-1,4,5,6,7,8-hexahydro-cycloheptapyrazole-3-carbonyl]-amino}-3-(4-fluoro-phenyl)-propionic acid methyl ester,
(8E)-8-(3-chloro-benzylidene)-1,4,5,6,7,8-hexahydro-cycloheptapyrazole-3-carboxylic acid [(1R)-2-hydroxy-1-phenyl-ethyl]-amide,
(8E)-(2S)-2-{[8-(3-chloro-benzylidene)-1,4,5,6,7,8-hexahydro-cycloheptapyrazole-3-carbonyl]-amino}-3-(4-fluoro-phenyl)-propionic acid methyl ester,
(8E)-(2S)-2-{[8-(3-fluoro-benzylidene)-1-methyl-1,4,5,6,7,8-hexahydro-cycloheptapyrazole-3-carbonyl]-amino}-3-(4-fluoro-phenyl)-propionic acid methyl ester,
(8E)-8-(3-fluoro-benzylidene)-1-methyl-1,4,5,6,7,8-hexahydro-cycloheptapyrazole-3-carboxylic acid [(1R)-2-hydroxy-1-phenyl-ethyl]-amide,
(8E)-(2S)-8-(3-fluoro-benzylidene)-1-methyl-1,4,5,6,7,8-hexahydro-cycloheptapyrazole-3-carboxylic acid [1-hydroxymethyl-2-(4-hydroxy-phenyl)-ethyl]-amide,
(8E)-(2R)-2-{[8-(3-chloro-benzylidene)-1,4,5,6,7,8-hexahydro-cycloheptapyrazole-3-carbonyl]-amino}-3-(4-fluoro-phenyl)-propionic acid methyl ester,
(8E)-(2R)-2-{[8-(4-chloro-benzylidene)-1,4,5,6,7,8-hexahydro-cycloheptapyrazole-3-carbonyl]-amino}-3-(4-fluoro-phenyl)-propionic acid methyl ester,
(8E)-8-(3-fluoro-benzylidene)-1-methyl-1,4,5,6,7,8-hexahydro-cycloheptapyrazole-3-carboxylic acid [(1R)-3-hydroxy-1-phenyl-propyl]-amide,
(8E)-8-(3-chloro-benzylidene)-1,4,5,6,7,8-hexahydro-cycloheptapyrazole-3-carboxylic acid [(1R)-3-hydroxy-1-phenyl-propyl]-amide,
(8R*)-(3-chloro-benzyl)-1,4,5,6,7,8-hexahydro-cycloheptapyrazole-3-carboxylic acid [(S)-2-methoxy-1-phenyl-ethyl]-amide,
(8S*)-(3-chloro-benzyl)-1,4,5,6,7,8-hexahydro-cycloheptapyrazole-3-carboxylic acid [(S)-2-methoxy-1-phenyl-ethyl]-amide,
(8S*)-(3-chloro-benzyl)-1,4,5,6,7,8-hexahydro-cycloheptapyrazole-3-carboxylic acid [(1R)-2-methoxy-1-phenyl-ethyl]-amide, and
(8R*)-(3-chloro-benzyl)-1,4,5,6,7,8-hexahydro-cycloheptapyrazole-3-carboxylic acid [(1R)-2-methoxy-1-phenyl-ethyl]-amide.

16. The method of claim 1, wherein the CB2 receptor mediated pain is chronic or acute.

17. The method of claim 16, wherein the CB2 receptor mediated pain is postoperative, inflammatory or neuropathic or the result of injury or age.

18. The method of claim 16, wherein the CB2 receptor mediated pain is a central or peripheral pathway mediated pain state that otherwise defies characterization and would benefit from treatment with a CB2 receptor agonist.

19. The method of claim 17, wherein the CB2 receptor mediated pain is inflammatory pain selected from the group consisting of osteoarthritis, rheumatoid arthritis, headache, migraine, odontaligia, labor, dysmenorrhea, interstitial cystitis, peripheral neuritis, mucositis, surgery pain, sports injury pain, trauma, cancer pain, fibromyalgia, pancreatitis, enteritis, cellulitis, bony fractures, post-operative ileus, irritable bowel syndrome, pain due to inflammatory bowel diseases, Crohn's Disease, ulcerative colitis, cholecystitis, burn, sunburn, pain due to venomous snake bite, spider bite or insect sting and pain due to nonvenomous snake bite, spider bite or insect sting.

20. The method of claim 17, wherein the CB2 receptor mediated pain is neuropathic pain selected from the group consisting of chemotherapeutic neuropathy, AIDS-related neuropathy, diabetic neuropathy and post herpetic neuralgia.

21. The method of claim 1, wherein the effective amount of the compound of claim 1 is from about 0.001 mg/kg/day to about 300 mg/kg/day.

22. The method of claim 1, wherein the effective amount of the compound of claim 13 is from about 0.001 mg/kg/day to about 300 mg/kg/day.

23. The method of claim 1, wherein the effective amount of the compound of claim 14 is from about 0.001 mg/kg/day to about 300 mg/kg/day.

24. The method of claim 1, further comprising administering to the subject a combination product and/or therapy comprising an effective amount of a compound of claim 1 and a therapeutic agent.

25. Use of the compound of claim 1 in the manufacture of a medicament for treating, ameliorating or preventing CB2 receptor mediated pain in a subject in need thereof.

26. The use of claim 25, wherein the CB2 receptor mediated pain is chronic or acute.

27. The use of claim 25, wherein the CB2 receptor mediated pain is postoperative, inflammatory or neuropathic or the result of injury or age.

28. The use of claim 25, wherein the CB2 receptor mediated pain is a central or peripheral pathway mediated pain state that otherwise defies characterization and would benefit from treatment with a CB2 receptor agonist.

29. The use of claim 27, wherein the CB2 receptor mediated pain is inflammatory pain selected from the group consisting of osteoarthritis, rheumatoid arthritis, headache, migraine, odontaligia, labor, dysmenorrhea, interstitial cystitis, peripheral neuritis, mucositis, surgery pain, sports injury pain, trauma, cancer pain, fibromyalgia, pancreatitis, enteritis, cellulitis, bony fractures, post-operative ileus, irritable bowel syndrome, pain due to inflammatory bowel diseases, Crohn's Disease, ulcerative colitis, cholecystitis, burn, sunburn, pain due to venomous snake bite, spider bite or insect sting and pain due to nonvenomous snake bite, spider bite or insect sting.

30. The use of claim 27, wherein the CB2 receptor mediated pain is neuropathic pain selected from the group consisting of chemotherapeutic neuropathy, AIDS-related neuropathy, diabetic neuropathy and post herpetic neuralgia.
